(19) United States
(12) Patent Application Publication
(10) Pub. No.: US 2012/0158099 A1
(43) Pub. Date: Jun. 21, 2012

(54) LOW LEVEL LASER THERAPY FOR LOW BACK PAIN

(75) Inventor: Elaine Lee, Santa Clara, CA (US)

(73) Assignee: KYPHON SARL, Neuchatel (CH)

(21) Appl. No.: 12/972,931

(22) Filed: Dec. 20, 2010

(51) Int. Cl. A61N 5/06

(52) U.S. Cl. 607/89

(57) ABSTRACT

The invention provides an energy source emitting, such as a low level laser light, catheter having a throughbore, an energy emitting surface disposed near the distal end of the catheter that is connected, directly or indirectly, to the energy source, which allows for the application of therapeutic energy to treat internal areas of a patient that have been accessed by way of a catheter.

### BACKGROUND

The present invention relates generally to an instrument to apply low level laser therapy to sites within a subject and methods of using the same.

Any publications or references discussed herein are presented to describe the background of the invention and to provide additional detail regarding its practice. Nothing herein is to be construed as an admission that the inventors are not entitled to antedate such disclosure by virtue of prior invention.

The intervertebral disc contains a nucleus pulposus surrounded by an annulus fibrosus. The nucleus pulposus provides the primary shock absorbing characteristics and the laminated nature of the annulus fibrosus provides the bulk of the tensile strength.

In a healthy intervertebral disc, cells within the nucleus pulposus produce an extracellular matrix (ECM) containing a high percentage of proteoglycans. These proteoglycans retain water, which provides at least some of the shock absorbing characteristics of the intervertebral disc. In at least some disc degeneration disease (DDD) situations there is a gradual degeneration of the intervertebral disc and a resulting decline in the amount of ECM present in the nucleus pulposus. In other instances of DDD, genetic factors, such as those leading to programmed cell death, or apoptosis can also cause the cells within the nucleus pulposus to emit deleterious amounts of cytokines and other deleterious proteins. In other instances, the pumping action of the disc may malfunction (due to, for example, a decrease in the proteoglycan concentration within the nucleus pulposus), thereby retarding the flow of nutrients into the disc as well as the flow of waste products out of the disc. All of these events are detrimental to the subject and the proper functioning of the disc.

There is a large amount of information indicating that red light irradiation of cartilage, nerves, and bone stimulates ECM production, decreases pain and stimulates bone production (see, US Pat. Pub. 2008/0221652, US Pat. Pub. 2007/0073363 and references cited therein). Therefore, low level laser therapy may be effective in the treatment of lower lumbar spinal pain using internally applied red light irradiation of the intervertebral disc, nerve roots and/or surrounding tissue. Traditionally, low level laser therapy (LLLT) has been applied from an external source. However, the lumbar spinal region is located deep beneath the skin, making external treatment largely ineffective. Therefore, there is a need in the art for the ability to apply LLLT beneath the skin, for example, to the lower lumbar region in a minimally invasive manner.

### SUMMARY OF THE INVENTION

Degeneration of the ECM (leading to decreased disc flexibility) is believed to benefit from the application of red light irradiation of the disc, which helps the disc regain its flexibility. Since there is evidence for anti-inflammatory effects of LLLT, as well as beneficial effects on fibroblast function and acceleration of connective-tissue repair, the treatment is believed to affect both the nerve endings surrounding the disc as well as the disc itself. In an exemplary embodiment, the red light has a wavelength of between 600 nm and 1000 nm. It is believed that red light having a wavelength of between 600 nm and 1000 nm delivered to the lumbar spine will provide a useful therapy to treat pain and/or degeneration of the disc, for example, in the lower lumbar region.

Therefore, in accordance with the present invention, there is provided a method of treating DDD and/or pain, comprising the step of: a) making a small incision in the skin of a subject; b) inserting a proximal end of a low level laser device into the incision; c) irradiating the intervertebral disc and/or area around it with an effective amount of red light having a wavelength of between 600 nm and 1,000 nm; and d) withdrawing the proximal end of the low level laser from the subject.

In another embodiment, there is also provided a method of treating DDD and/or pain, comprising the step of: a) making a small incision in the skin of a subject; b) inserting a proximal end of a low level laser device into the incision; c) irradiating the intervertebral disc and/or area around it with an effective amount of red light having a wavelength of between 600 nm and 1,000 nm; d) injecting a beneficial substance into the intervertebral disc and/or area around it; and e) withdrawing the proximal end of the low level laser from the subject.

In another exemplary embodiment, there is provided a method of treating pain in or around the spine, comprising the step of: a) making a small incision in the skin of a subject; b) inserting a proximal end of a low level laser device into the incision; c) irradiating an area in or around the spine, such as a facet joint, a vertebra, and/or a nerve root with an effective amount of red light having a wavelength of between 600 nm and 1,000 nm; and d) withdrawing the proximal end of the low level laser from the subject. Optionally, a mixture of local anaesthetic and a corticosteroid (e.g., 17-hydroxy-11-dehydrocorticosterone) may be injected through a catheter.

In an exemplary embodiment, the intervertebral disc, facet joint, vertebra, nerve root and/or surrounding tissue is irradiated with between about 0.02 J/cm² to about 200 J/cm², between about 0.2 J/cm² to about 50 J/cm², or between about 1 J/cm² to about 10 J/cm² of energy received at the desired tissue. In another exemplary embodiment, the laser is powered by between about 5 milliwatts and about 500 milliwatts.

Methods and devices of the present invention generally facilitate treatment of pain, e.g., discogenic pain, spinal pain and/or injury, due to a surgical procedure, such as inserting a catheter into a subject. In an exemplary embodiment, methods and devices of the invention help treat one or more intervertebral discs, facet joints, vertebra, and/or nerve roots in a patient. In one embodiment, a distal portion of a catheter device is positioned in an intervertebral disc that is thought to be the cause of a subject's pain and a therapeutic energy is applied to the disc and/or tissue around the disc. One or more anchoring members may be used to maintain the distal portion of a catheter in the disc and the distal portion and/or the one or more anchoring members of the catheter may be used to apply a therapeutic energy, such as LLLT, ultrasound, vibrational energy, radio frequency and/or thermal energy. The therapeutic energy may be applied to the annulus fibrosis, nucleus pulposus and/or tissue adjacent to a disc. One or more substances, such as an antibiotic, therapeutic agent or analgesic, may also be injected into or around the disc in conjunction with the delivery of the therapeutic energy. Catheter devices of the invention generally include one or more anchoring members for maintaining a distal portion of the catheter device in a position within a disc and diffusing the therapeutic energy into the surrounding area. The catheter device may also be coupled with, for example, an implantable pump or injection port, and the pump or port may be used to supply one or more substances, such as antibiotic, therapeutic agent or analgesic, to the disc to treat a patient's back pain.

In an exemplary embodiment the invention provides a method for introducing one or more substances and/or therapeutic energies into an intervertebral disc by positioning a distal portion of a catheter device in the disc, anchoring the distal portion of the catheter device to maintain the distal portion in the disc, and introducing at least one therapeutic energy into the disc through the catheter device. A number of different methods for positioning the distal portion may be employed in various embodiments of the invention. In one embodiment, for example, the catheter device is passed through a lumen of an introducer device. In one such embodiment positioning the distal portion of the catheter involves passing the catheter device through the lumen of the introducer device over a pointed stylet, piercing through an annulus fibrosis of the disc using the stylet, and withdrawing the stylet from the catheter device. In an alternative embodiment, positioning the distal portion involves piercing through an annulus fibrosis of the disc into the disc with a tapered distal end of the catheter device. In other embodiments, the catheter device is passed over a guidewire. See U.S. Pat. No. 7,452,351, the entirety of which is hereby incorporated by reference.

In some embodiments, anchoring the catheter device involves deploying one or more anchoring members disposed along the catheter body. In some cases, such anchoring members may be disposed at or near the distal portion of the catheter, while in other embodiments anchoring may occur at locations farther from the distal portion. In one embodiment, anchoring involves inflating at least a first expandable member in the disc. Optionally, this technique may further involve inflating at least a second expandable member adjacent an outer surface of the disc, such that there is one inflatable member in the disc and another inflatable member just outside the annulus fibrosis. In one embodiment, the first and/or second expandable member is coupled to a therapeutic energy source, such as LLLT, and adapted to deliver the therapeutic energy source to surrounding tissue. In one embodiment, the therapeutic energy source is a red light source and the first and/or second expandable members diffuse the red light into the tissue surrounding the first and/or second expandable members.

The catheter device and introducer device may have any suitable dimensions, but in one embodiment the outer diameter of the catheter body is less than 2 mm. An inner diameter of the needle, in some embodiments, is between about 0.1 mm and about 0.01 mm larger than the outer diameter of the catheter body.

In another embodiment, anchoring the catheter comprises deploying at least one mechanism located at or near the distal portion of the catheter device to increase the effective cross-sectional diameter of the catheter at one or more locations. For example, the cross-sectional diameter may be increased by releasing one or more shape memory or spring loaded members from constraint. In other embodiments, the cross-sectional diameter may be increased by actuating one or more mechanical members or moving an inner catheter shaft of the catheter device relative to an outer catheter shaft of the catheter device to cause one or more anchoring members to buckle outwards. In other exemplary embodiments, the anchoring members are configured to transmit the therapeutic energy, e.g., LLLT, to the surrounding tissue. In another exemplary embodiment the anchoring members comprise a light carrying membrane connected to a LLLT light source located away from the distal end of the catheter and configured to diffusely apply LLLT treatment to the surrounding tissue.

In another exemplary embodiment, the catheter device may either be advanced to a position within the disc or to a position just outside the disc. In either case, the catheter device may have a distal end or portion configured to facilitate advancement of the distal end through the annulus fibrosis. In some embodiments, the catheter device is passed over a guidewire. In some cases, the catheter is passed over the guidewire within the needle, while in alternative embodiments an introducer is removed over the guidewire before the catheter device is passed over the guidewire. In some embodiments, positioning the distal portion is facilitated by visualizing at least one radiopaque marker or material at or near the distal portion to assess its location.

The one or more substances introduced into the disc may be any suitable substance, typically introduced for diagnosis and/or treatment of discogenic pain, but in alternative embodiments for any other suitable purpose. Any suitable combination of substances may be introduced, either simultaneously or sequentially, for diagnosis, treatment or other purposes. In some embodiments, one or more placebo substances may be introduced into one or more discs, typically to assist in diagnosis but in other embodiments for study or experimental purposes or the like. In some embodiments, for example, introduced substance(s) may include, but are not limited to; an analgesic; an antibiotic; or a therapeutic agent such as: a hydrating agent such as hypotonic saline, isotonic saline or hypertonic saline; a supportive agent such as a hydrogel, ethylene-vinyl alcohol copolymer, dimethyl sulfoxide or tantalum; a prolotherapy agent such as sodium morrhuate, cod oil, phenol, minerals or ethyl alcohol; and other agents such as collagen, stem cells, Osteogenic Protein-1, ethanol, alcohol, steroids, radio-opaque contrast agents, ultrasound contrast agent, Bone Morphogenetic Protein (BMP), BMP-2, BMP-4, BMP-6, BMP-7, BMP-12, Serotonin 5-HT2A receptor inhibitors, TNF inhibitors, LMP-1, TIMP-1, TGF-1, TGF-2, Rofecoxib, Ketorolac, Glucosamine, Chondroitin Sulfate, Dextrose, DMSO, non-steroidal antiinflammatory drugs, ibuprofen, naprosyn, Bextra, Vioxx, Celebrex, indomethacin, botulinum toxin, capsaicin, vanilloid agonists, vanilloid antagonists, VR1, VRL-1, methylprednisolone and/or chemonucleolysis agents such as chondroitinase ABC, hyaluronidase, polylysine, or chymopapain.

Examples of antibiotics and analgesics include, but are not limited to, lidocaine, chloroprocaine, mepivacaine, ropivacaine, xylocaine, prilocaine, morphine, bupivocaine, marcaine, 2-chloroprocain, fentanyl, diamorphine, meperidine, methadone, alfentanil, hydromorphone, lofentanil, sufentanil, buprenorphine, other opoids, adrenergic agonists, somatostatin analogs, calcium channel blockers, N-methyl-D-aspartate receptor antagonists, ketamine, benzodiazepines, klonidine, tizanidine, midazolam, levorphanol, heterocyclic antidepressants, nonheterocyclic, serotonin-enhancning antidepressants, GABA analogues, psychogenic amines, somatostatin, octreotide, SNX-111, midazolam, methylprednisolone acetate, Aristospan, ethyl chloride, etidocaine, linocaine, triamcinolone diacatate, Astramorph, Duramorph, Dilaudid, Sensorcaine MPF, Baclofen (Lioresal), Clonidine, baclofen, codeine, neurontin and Demerol. Examples of antibiotics include, but are not limited to, Penicillins, Cephalosporins, Tetracycline, Erythromycin, Clindamycin, Vancomycin, Bacitracin, Doxycycline, Ampicillin, Levaquin, Metronidazole, Azithromycin, Ciprofloxacin, Augmentin, Bactrim, TMP-SMX, Rocephin, Gentamycin, Keflex and Macrobid.

In some embodiments, the method further involves, before introducing the substance, applying the therapeutic energy. In such embodiments, the substance introduced is an analgesic, which may be used in determining whether the patient feels the spinal pain after introduction of the substance to help determine whether pain is caused by that particular disc. Alternatively, applying the therapeutic energy may be done after introducing the substance and/or after determining whether pain is caused by that particular disc. The method may also involve, before introducing the at least one substance into the disc, causing the patient to assume a position in which substantial spinal pain is experienced. In some embodiments, the method may optionally further include performing a discography procedure on the intervertebral disc before or after positioning the distal portion of the catheter device in the disc.

Some embodiments of the method further include leaving the catheter device in position with the distal portion in the disc and administering the at least one substance over time and periodically applying therapeutic energy to provide treatment of spinal pain. As described herein, in some embodiments the substance(s) may be administered over time via a subcutaneous injection port or implanted pump and/or subcutaneous therapeutic energy source, the method further comprising coupling the catheter device to the subcutaneous therapeutic energy source and subcutaneous injection port or implanted pump. In alternative embodiments, the substance(s) may be administered over time via any other suitable combination of devices or other means.

In yet another exemplary embodiment, a catheter device for applying therapeutic energy and/or introducing one or more substances into a subject comprises an elongate flexible catheter body and at least one anchoring member disposed along the catheter body for anchoring at least part of the distal portion of the catheter into a fixed position within the subject. The catheter body itself has a proximal portion, a distal portion for delivering therapeutic energy, at least one lumen for introducing one or more substances into the subject at or near an area thought to be causing pain and at least one therapeutic energy delivery surface located at or near the distal portion of the catheter.

In some embodiments, the anchoring member is disposed on or near the distal portion of the catheter, while in other embodiments it may be located farther from the distal portion. In other embodiments, the therapeutic energy delivery surface is located at or near the distal portion of the catheter and configured to diffuse an infrared light energy into surrounding tissue. The anchoring member (or multiple anchoring members) and therapeutic energy delivery surface (or multiple therapeutic energy delivery surfaces) of the catheter device may take any of a number of various forms. For example, in one embodiment the anchoring member and therapeutic energy delivery surface comprise at least one expandable member coupled with an inflation lumen and a therapeutic energy source. In an alternative embodiment, the anchoring member comprises at least one shape memory, spring loaded or mechanically activated member for increasing the effective cross-sectional diameter of the catheter body at a first position at or near the distal portion and the therapeutic delivery surface is located at a second position near the distal portion.

In an exemplary embodiment, the anchoring member may comprise at least one outwardly buckling member coupled with an inner catheter shaft and an outer catheter shaft of the catheter body so as to outwardly buckle when the inner shaft is moved axially relative to the outer shaft, wherein the at least one outwardly buckling member comprises at least one therapeutic delivery surface.

In another exemplary embodiment, the anchoring member comprises at least one deformable member made of a light transmitting material to change at least part of the distal portion from a substantially straight shape to a substantially curved or geometric shape, wherein the deformable member is directly or indirectly connected to an inflation lumen and a therapeutic energy source.

Optionally, the catheter body may also include an outer surface having one or more markings for indicating depth of insertion of the catheter device into a patient's body. In other embodiments, the catheter body may include an outer surface having two or more different colors for indicating depth of insertion of the catheter device into a patient's body. The catheter body may further include at least one radiopaque marker or material for facilitating visualization of the catheter device in a patient.

In an exemplary embodiment, the catheter device includes an injection tube extending through at least part of the lumen of the catheter body for introducing one or more substances into the disc, an inflation tube extending through at least part of the lumen for expanding the deployable anchoring member and a therapeutic energy conduit extending through at least part of the lumen for delivering therapeutic energy to a therapeutic delivery surface. The catheter body, therapeutic energy conduit and/or injection tube may be made of any suitable material or combination of materials, such as but not limited to, stainless steel, tempered stainless steel, annealed stainless steel, polymers, flexible plastics, optical fiber, an electrical wire or conduit, superelastic alloys and/or other suitable materials. In some embodiments, the therapeutic energy conduit, injection tube and/or inflation tube exit a proximal end of the catheter body and are removably coupled with at least one adapter to provide for application of therapeutic energy, injection and inflation. In some embodiments, the therapeutic energy conduit, injection tube and inflation tube extend through at least part of the catheter body lumen coaxially. Alternatively, the therapeutic energy conduit, injection tube and inflation tube may extend through at least part of the catheter body lumen side-by-side. In other embodiments, the therapeutic energy conduit, injection tube and inflation tube may extend through part of the catheter body lumen coaxially and through another part of the lumen side-by-side. In an alternative embodiment, the catheter body comprises the therapeutic energy conduit and may optionally include one or more surface coatings to prevent discharge of the energy in undesired locations along the catheter body. In another embodiment, the catheter comprises a single extrusion having an injection lumen for introducing one or more substances into the disc and an inflation lumen for expanding the at least one anchoring member, with the therapeutic energy conduit located within the lumen, within the catheter body or outside of the catheter body.

In an exemplary embodiment, the catheter device includes an injection tube extending through at least part of the lumen of the catheter body for introducing one or more substances into the disc, an inflation tube extending through at least part of the lumen for expanding the deployable anchoring member and a therapeutic energy conduit comprising an electrical conduit extending through at least part of the lumen and connected to an infrared light at or near the end of the catheter, wherein the infrared light is configured to deliver infrared therapeutic energy to a therapeutic delivery surface on the catheter. In some embodiments, the therapeutic energy conduit, injection tube and/or inflation tube exit a proximal end of the catheter body and are removably coupled with at least one adapter to provide for application of therapeutic energy via the electrical conduit and infrared light, injection of a substance and inflation of the anchoring member. Alternatively, the therapeutic energy conduit, injection tube and inflation tube may extend through at least part of the catheter body lumen side-by-side. In another embodiment, the catheter comprises a single extrusion having an injection lumen for introducing one or more substances into the disc and an inflation lumen for expanding the at least one anchoring member, with the therapeutic energy conduit located within the lumen, within the catheter body or outside of the catheter body.

In various embodiments, the catheter device may have any suitable proximal end configurations for providing connection to one or more therapeutic energy sources, injection, inflation, suction, irrigation or other devices, for providing guidewire access and/or the like. In one embodiment, for example, a proximal end of the proximal portion of the catheter body is trifurcated into three separate catheter body proximal ends. In some embodiments, each of the three proximal ends is removably coupled with an adapter for facilitating energy delivery, injection or inflation.

In another exemplary embodiment, a system for introducing one or more therapeutic energies and one or more substances into an intervertebral disc includes an introducer device and a catheter device passable through the introducer device. The catheter device includes an elongate flexible catheter body and at least one deployable anchoring member disposed along the catheter body for anchoring at least part of the distal portion of the catheter in the disc. The catheter body includes a proximal portion, a self-introducing distal portion for facilitating penetration of an annulus fibrosis of the disc, and at least one lumen for introducing one or more substances into the intervertebral disc. In various embodiments, the catheter device may include any of the features, configurations or combinations described above.

In some embodiments, the catheter system may further include an automatic energy source device removably coupled with the catheter device for automatically delivering an appropriate amount of therapeutic energy into the disc, to the annulus and/or surrounding soft tissue.

### BRIEF DESCRIPTION OF THE FIGURES

FIG. 1A-1K illustrate a method for positioning a catheter device to introduce a substance into an intervertebral disc, shown from a transverse cross-section of the spinal column, according to one embodiment of the present invention.

FIGS. 2A and 2B illustrate part of a method for positioning a catheter device to introduce a substance and a therapeutic energy into an intervertebral disc using a pointed stylet, according to one embodiment of the present invention.

FIG. 2C illustrates a catheter device in place for introducing a substance and therapeutic energy into an intervertebral disc and an introducer device being split, according to another embodiment of the present invention.

FIGS. 3A and 3B are perspective and cross-sectional views, respectively, of a distal end of a catheter device, according to one embodiment of the present invention.

FIGS. 4A and 4B are cross-sectional views of a distal end of a catheter device with an anchoring member in an undeployed and deployed state, respectively, according to one embodiment of the present invention.

FIGS. 5A and 5B are cross-sectional views of a distal end of an alternative catheter device with an anchoring member in an undeployed and deployed state, respectively, according to another embodiment of the present invention.

FIGS. 6A and 6B are cross-sectional views of a distal end of an alternative catheter device with an anchoring member in an undeployed and deployed state, respectively, according to another embodiment of the present invention.

FIGS. 7 A and 7B are perspective views of a distal end of an alternative catheter device with an anchoring member in an undeployed and deployed state, respectively, according to another embodiment of the present invention.

FIG. 8 illustrates a catheter device having two anchoring members and therapeutic energy delivery surfaces for anchoring and applying therapeutic energy inside and outside an annulus fibrosis of an intervertebral disc, according to one embodiment of the present invention.

FIGS. 9A and 9B are perspective and cross-sectional views, respectively, of a proximal adapter for use with a catheter device, according to one embodiment of the present invention.

### DETAILED DESCRIPTION OF THE INVENTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications in the illustrated device, and such further applications of the principles of the invention as described herein being contemplated as would normally occur to one skilled in the art to which the invention relates.

The uses of the terms “a” and “an” and “the” and similar references in the context of describing the invention (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “comprising,” “including,” “containing,” “characterized by,” and grammatical equivalents thereof are inclusive or open-ended terms that do not exclude additional, unrecited elements or method steps, but will also be understood to include the more restrictive terms “consisting of” and “consisting essentially of.”

In an exemplary embodiment, the therapeutic energy is a red light (LLLT) that may be delivered to the disc, including the annulus fibrosis and nucleus pulposus, facet joint or a vertebra. This mode of delivery allows the clinician to apply the therapeutic benefits of LLLT directly to the disc, including the inner part of the disc. In another preferred embodiment, the LLLT is delivered into at least the nucleus pulposus following a disc biopsy, laminaplasty, laminectomy, spinal fusion, treatment of a herniated disc, or other such treatments. Delivery of LLLT following such treatments can be beneficial in aiding wound healing due to puncturing the disc.

A number of mechanisms have been proposed for how LLLT produces beneficial results. Without wishing to be bound by theory, LLLT may increase ATP production by the mitochondria and increased oxygen consumption on the cellular level; increase serotonin and endorphins; reduce prostaglandin synthesis; increase lymphatic flow; decrease inflammation; and/or decrease edema.

In other exemplary embodiments, the therapeutic energy is delivered to the outside surface of the annulus fibrosus of the disc, the surface of the puncture site and/or into the nucleus pulposus. In another exemplary embodiment, the therapy is delivered by emitting the therapeutic energy during insertion and/or removal of the distal end of the catheter from the nucleus pulposus.

In an exemplary embodiment, the invention provides a method of analyzing and treating an interverterbral disc by inserting a catheter into a vertebral body wherein the catheter comprises a therapeutic delivery or discharging surface that is an energy transmitting surface, e.g., an optical fiber, as a therapeutic conduit, e.g., fiber optic cable, connected to a therapeutic energy source to transmit the therapeutic energy, e.g., red light, from the energy source to the transmission surface and emit it in the intervertebral disc, e.g., into the nucleus pulposus. In addition, one or more substances may be delivered through the catheter or a biological sample may be withdrawn from the catheter in combination with the application of the therapeutic energy. This allows for the application of therapeutic energies and one or more of adding a beneficial substance and/or removing a biological sample.

In another exemplary embodiment, the therapeutic energy is Low Level Laser Therapy (LLLT) using red light having a wavelength between about 600 and about 100 nm. In this embodiment, the distal end portion of the catheter comprises one or more light emission surfaces configured to transmit the red light into the surrounding tissue. The therapeutic energy conduit is optionally substantially transparent to the red light and acts as a pathway for red light that is eventually emitted at or near the distal end of the catheter or an electrical conduit that connects a power source external to the subject to a light source located at or near the distal end of the catheter. In some embodiments, the therapeutic energy conduit is a translucent material that comprises a flexible polymer, which may be in the form of coatings, long fibers and/or combinations thereof. Suitable red light transmissible polymers are preferably selected from the group consisting of polypropylene and polyesters.

The catheter system of the invention includes an externally based control device having an therapeutic energy source, such as a LLLT light source or electrical power source, wherein the therapeutic energy is transmitted along the length of the catheter by an appropriate therapeutic energy conduit, such as a fiber optic cable or electrical wire for LLLT, and connected to a therapeutic delivery surface located on or near the distal end of the catheter. The therapeutic delivery surface being in appropriate communication with the therapeutic energy conduit and configured to deliver the therapeutic energy to the surrounding tissue in a controllable and uniform way.

It should be noted that the catheter of the present invention is not a simple metal tube, e.g., filled with silica or other light transmitting substance, that receives light and transmits it to adjacent tissue, for example, cancellous tissue.

In an exemplary embodiment, the therapeutic energy delivery surface or an area substantially adjacent thereto may have sensor adapted to detect disc tissue, annulus fibrosis and/or nucleus pulposus tissue, and activate the therapeutic energy source or activate transmission of the therapeutic energy to the tissue Likewise, the therapeutic energy source may be configured to deliver a predetermined amount of energy, which may be an amount determined by the treating physician or preset by the manufacturer.

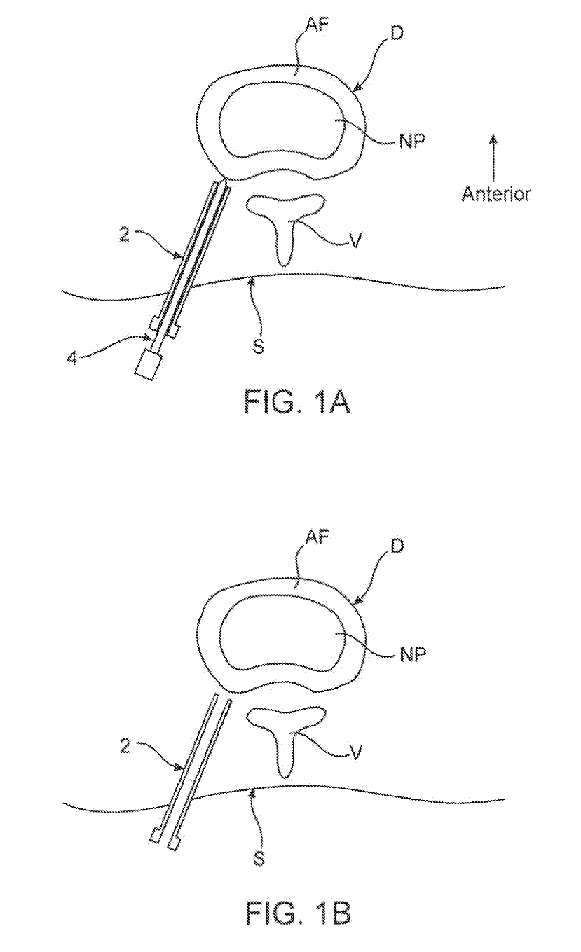

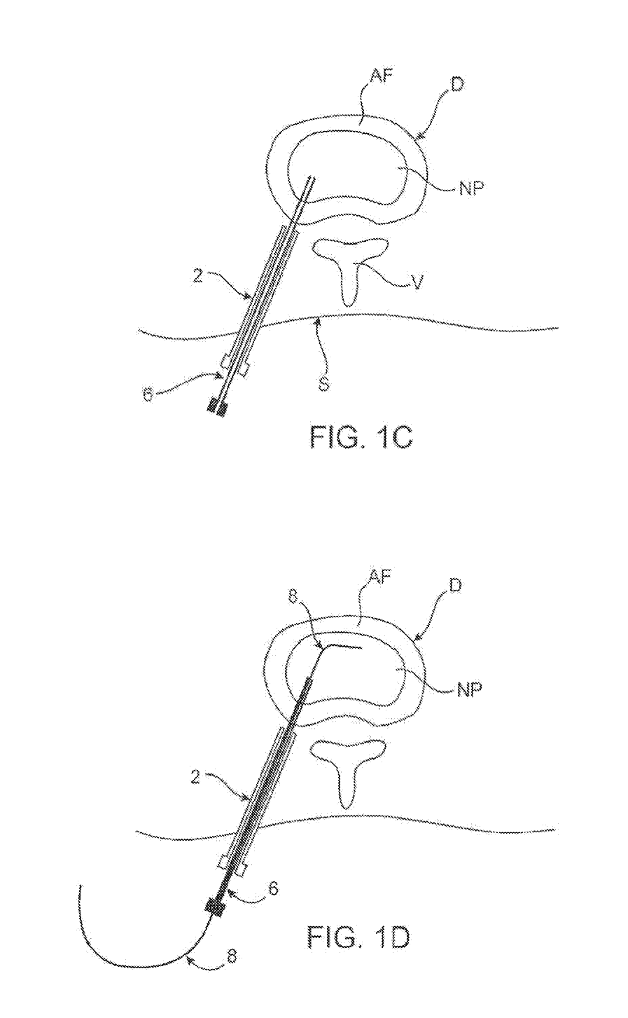

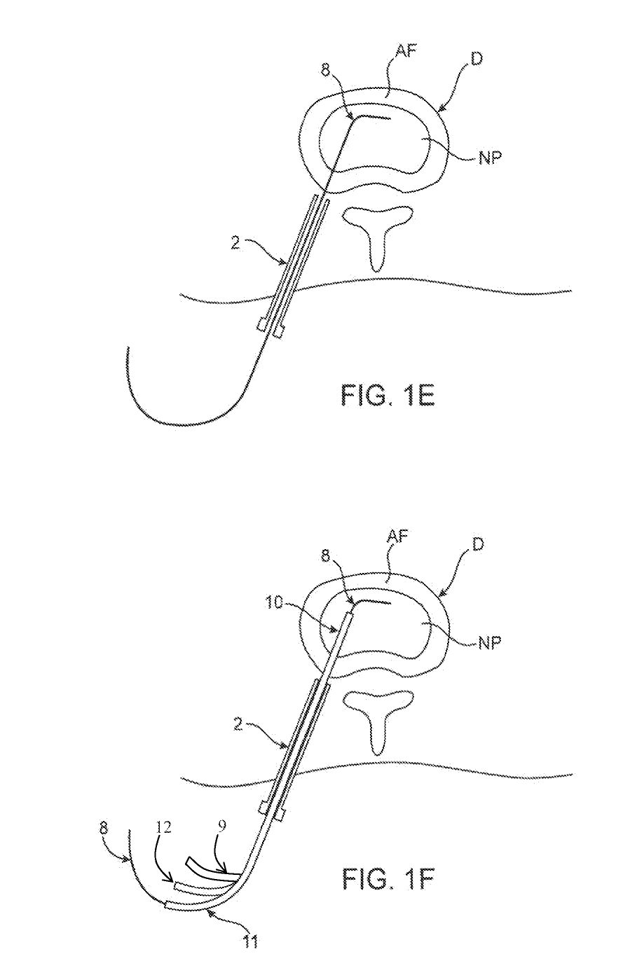

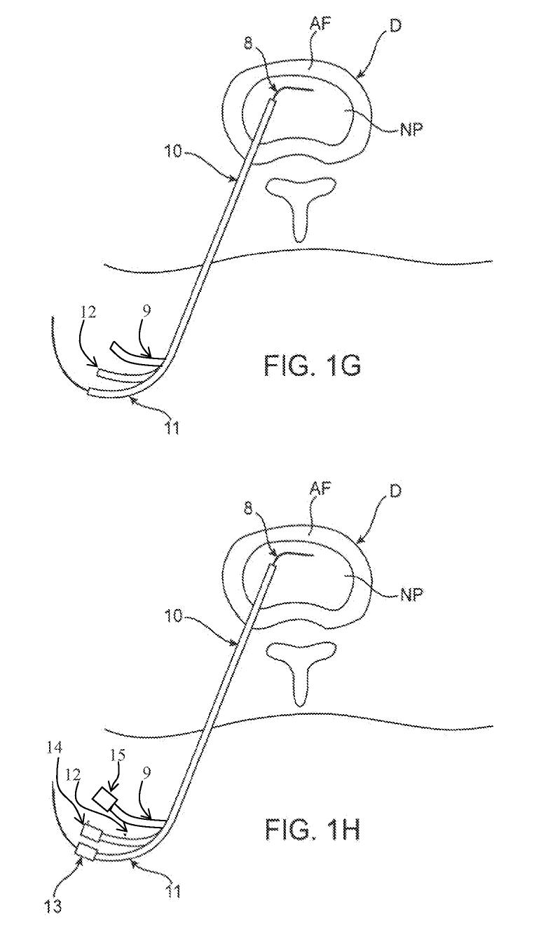

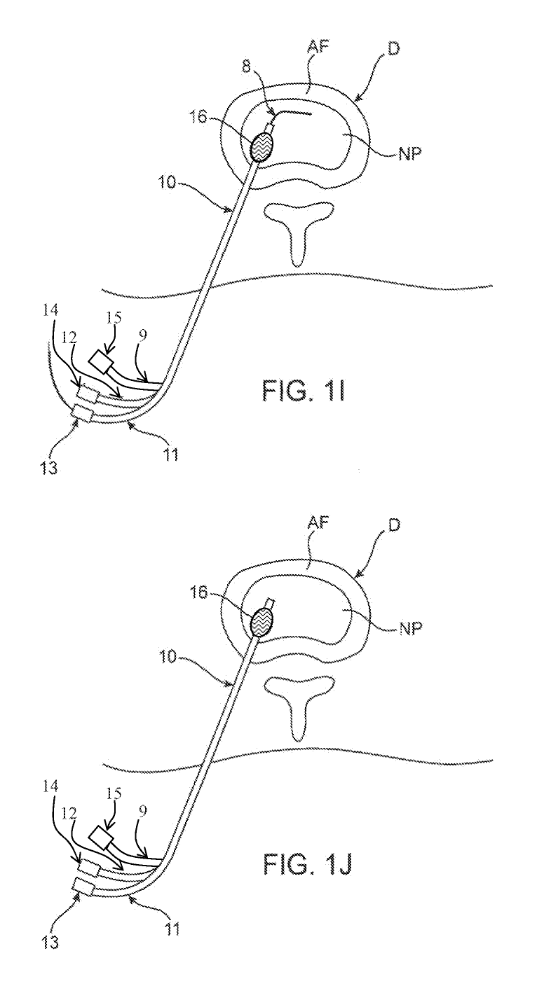

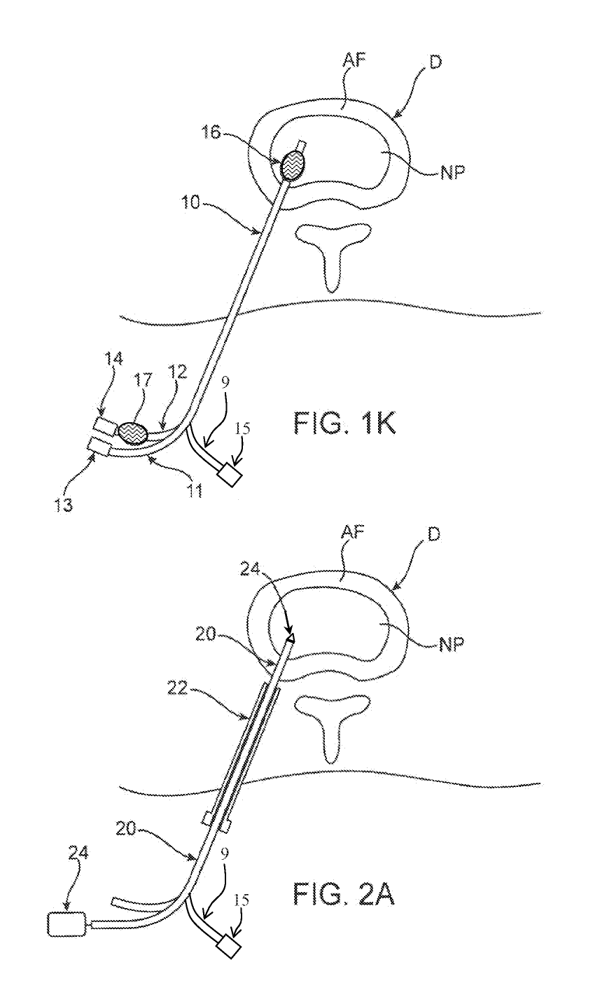

Referring now to FIGS. 1A-1K, a method for introducing a substance and a therapeutic energy into an intervertebral disc is illustrated schematically. As seen in FIG. 1A, an intervertebral disc D includes an annulus fibrosis AF surrounding a nucleus pulposus NP, and is positioned adjacent a spinous process of a vertebra V. Anatomically, the disc D is sandwiched between two vertebrae of the spine (not shown), which lie on top of and beneath the disc D.

In one embodiment, an introducer device 2 and a pointed obturator 4 are introduced together through the skin S of a patient's back to position their distal ends near the intervertebral disc D. Introducer device 2 and obturator 4 may have any suitable dimensions, but in one embodiment introducer device 2 is about 18-22 gauge and obturator 4 is about 20-25 gauge.

As shown in FIG. 1B, obturator 4 is then removed, leaving introducer device 2 in place. As shown in FIG. 1C, an injection needle 6 is then passed through introducer device 2 and through the annulus fibrosis AF to position its distal tip in the nucleus pulposus NP. Position of introducer device 2 and/or injection needle 6 may be confirmed using x-ray, fluoroscopy, or other suitable means. In some embodiments, when injection needle 6 is positioned in the nucleus pulposus NP, contrast dye may be injected through injection needle 6, and the appearance of the contrast dye in the disc as well as the patient's response to the injection may be monitored. This part of the procedure generally describes a known discography procedure. In alternative embodiments, discography may be performed at a later time or no discography may be performed.

After placing injection needle 6, a guidewire 8 may be passed through injection needle 6 into the disc, as shown in FIG. 1D. Injection needle 6 may then be removed, as shown in FIG. 1E, and a catheter device 10 may be passed over guidewire 8 through introducer device 2, as shown in FIG. 1F. Catheter device 10 is described in further detail below, but in one embodiment it may include two or more tubes, such as a guidewire tube 11, an injection or inflation tube 12 and a therapeutic energy conduit 9, such as an fiber optic cable, which may separate proximally to attach to multiple adapters or the like. Once catheter device 10 is in place, introducer device 2 may be removed, as shown in FIG. 1G, and adapters 13, 14 and 15 may be coupled with the proximal ends of conduit 9 and tubes 11 and 12, as shown in FIG. 1H. Adapters 13, 14 and 15 may facilitate guidewire passage, inflation of an expandable member, injection of one or more substances into the disc and transmission of a therapeutic energy, such as LLLT and/or the like.

Referring to FIG. 1I, one or more anchoring members 16 disposed along catheter 10 are deployed to maintain a distal portion of catheter in the disc. In one embodiment, anchoring member 16 comprises an expandable balloon that is also a therapeutic delivery surface, but as is described in more detail below, many other types of anchoring members may be used in various alternative embodiments. As shown in FIG. 1J, once anchoring member 16 is deployed, guidewire 8 may be removed. In some embodiments, as in FIG. 1K, a marker expandable member 17 may be deployed outside the patient's body. With anchoring member 16 and thus the distal portion of catheter 10 in place in the disc, therapeutic energy and one or more substances are introduced into the disc through catheter 10.

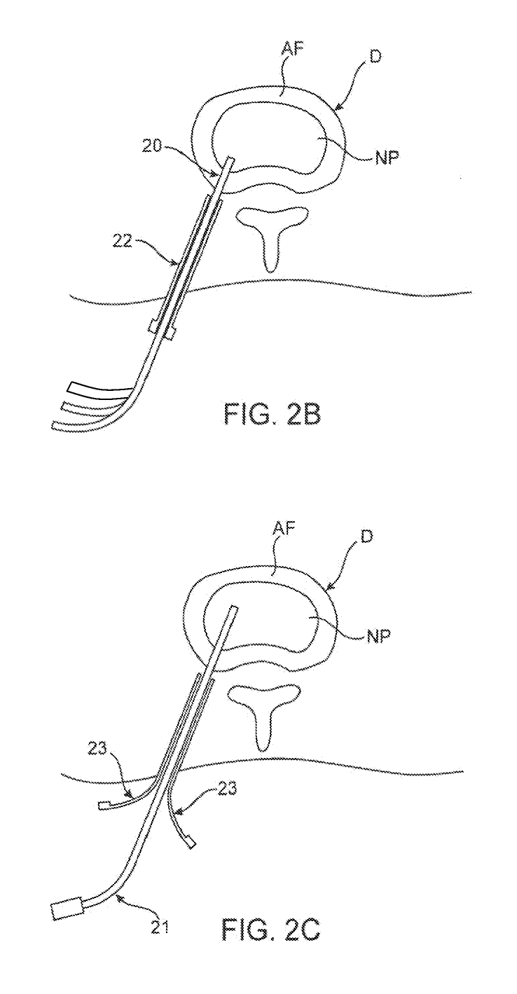

The method just described is but one embodiment of a technique for placing and anchoring a distal portion of a catheter within a disc and introducing a substance and a therapeutic energy therein. In various alternative embodiments, any number of suitable changes to the technique, such as additions or deletions of various steps, use of varied devices and the like, may be made without departing from the scope of the present invention. FIGS. 2A and 2B illustrate part of an alternative embodiment of a method for passing a catheter device 20 into a disc for introducing therapeutic energy and one or more substances. In this embodiment, rather than employing a guidewire passed through an injection needle, catheter device 20 is passed through an introducer device 22 with a pointed stylet 24 extending through a lumen of catheter 20 and out its distal tip. Stylet 24 enables catheter 20 to be passed through the tough annulus fibrosis AF without the help of a guidewire and injection needle. Stylet 24 is then removed, as shown in FIG. 2B, to leave catheter 20 in position for anchor deployment and introduction of therapeutic energy and a substance. Referring now to FIG. 2C, a method for introducing a catheter device 21 into a disc to inject one or more substances may be facilitated in one embodiment through use of a split away introducer device 23. Split-away introducer device 23 may be used just as the introducer devices have been described above. Rather than removing split-away introducer device 23 by sliding it off the proximal end of catheter device 21, however, split-away introducer device 23 is split along its length to be removed from catheter device 21. Split-away introducer device 23 may be constructed from any suitable material or materials so as to readily tear, crack, fissure, rip or separate along the length of needle 23. Splitting may be accomplished by including a perforation, thin section or other weakness along the length of needle 23.

Typically, once a catheter device is in place, with a distal portion residing in a disc and one or more anchoring members are deployed to maintain the catheter's position, the patient is instructed to assume a position or perform a task that typically causes the patient pain, such as bending over to pick up an object or the like. Therapeutic energy and/or a substance is then introduced into the disc, and the patient is asked to relate whether pain is lessened, eliminated, remains the same or the like. In various embodiments, the patient is asked to rate the experienced pain on a scale of 1 to 10 before and after introduction of the therapeutic energy and/or substances into the disc. In one embodiment, the therapeutic energy is LLLT and/or the substance introduced is an analgesic, and thus may alleviate the patient's pain when applied to or injected into the disc that is actually causing the pain.

In some instances multiple applications of the therapeutic energy and/or substances are performed. Again, such testing may be performed either alone or before or after traditional discography.

In some embodiments, multiple discs of one patient may be accessed and tested. Also in some embodiments, testing may be performed over a prolonged period of time, to test multiple discs and/or to enhance the accuracy or certainty of test results. In each case therapeutic energy, such as LLLT may be applied to help reduce an inflammatory response to the catheter 21.

In various embodiments, any of a number of different substances may be introduced into a disc. For different purposes, such as diagnosis or treatment of discogenic pain, study purposes or experimentation or the like, introduction of different therapeutic energies and/or substances may be warranted.

Examples of possible therapeutic energies that may be introduced into a disc include, but are not limited to, LLLT, ultrasound, vibrational energy, radio frequency, electrical stimulation, and/or thermal energy.

Examples of possible substances that may be introduced into a disc include, but are not limited to anesthetics; analgesics; antibiotics; hydrating agents such as hypotonic saline, isotonic saline or hypertonic saline; supportive agents such as a hydrogel, ethylene-vinyl alcohol copolymer, Dimethyl Sulfoxide or Tantalum; prolotherapy agents such as sodium morrhuate, cod oil, phenol, minerals or ethyl alcohol; and/or other agents such as collagen, stem cells, Osteogenic Protein-I, ethanol, alcohol, steroids, radio-opaque contrast agents, ultrasound contrast agent, Bone Morphogenetic Protein (BMP), BMP-2, BMP-4, BMP-6, BMP-7, BMP-12, Serotonin 5-HT2A receptor inhibitors, TNF inhibitors, LMP-I, TIMP-I, TGF-I, TGF-2, Rofecoxib, Ketorolac, Glucosamine, Chondroitin Sulfate, Dextrose, DMSO, non-steroidal antiinflammatory drugs, ibuprofen, naprosyn, Bextra, Vioxx, Celebrex, indomethacin, botulinum toxin, capsaicin, vanilloid agonists, vanilloid antagonists, VRI, VRL-I, methylprednisolone or, or chemonucleolysis agents such as chondroitinase ABC, hyaluronidase, polylysine, chymopapain.

Examples of antibiotics and analgesics include, but are not limited to lidocaine, chloroprocaine, mepivacaine, ropivacaine, xylocaine, prilocaine, morphine, bupivocaine, marcaine, 2-chloroprocain, fentanyl, diamorphine, meperidine, methadone, alfentanil, hydromorphone, lofentanil, sufentanil, buprenorphine, other opoids, adrenergic agonists, somatostatin analogs, calcium channel blockers, N-methylD-aspartate receptor antagonists, ketamine, benzodiazepines, klonidine, tizanidine, midazolam, levorphanol, heterocyclic antidepressants, nonheterocyclic, serotonin-enhancning antidepressants, GABA analogues, psychogenic amines, somatostatin, octreotide, SNX-III, midazolam, methylprednisolone acetate, Aristospan, ethyl chloride, etidocaine, linocaine, triamcinolone diacatate, Astramorph, Duramorph, Dilaudid, Sensorcaine MPF, Baclofen (Lioresal), Clonidine, baclofen, codeine, neurontin and Demerol. Examples of antibiotics include, but are not limited to, Penicillins, Cephalosporins, Tetracycline, Erythromycin, Clindamycin, Vancomycin, Bacitracin, Doxycycline, Ampicillin, Levaquin, Metronidazole, Azithromycin, Ciprofloxacin, Augmentin, Bactrim, TMP-SMX, Rocephin, Gentamycin, Keflex and Macrobid.

As already mentioned, in some embodiments the method further includes leaving the catheter device in place to provide treatment of a patient's back pain, such as continued or periodic application of the therapeutic energy or injection of a beneficial substance. In some embodiments, the catheter device may be coupled with an implantable pump, injection port or other device to provide such treatment.

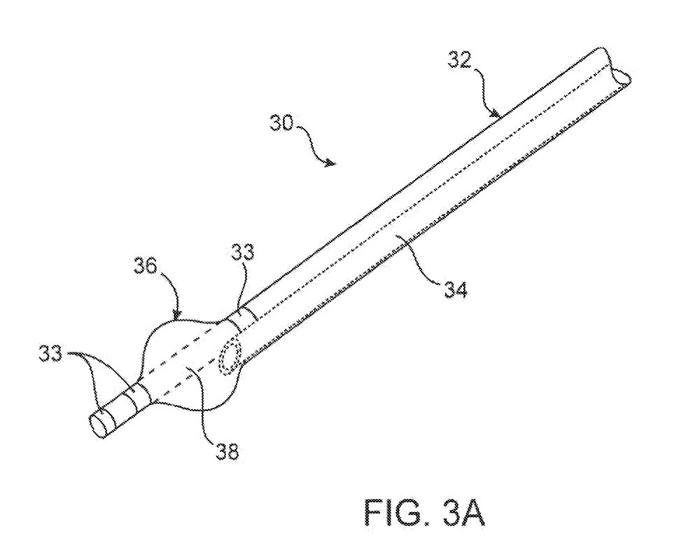

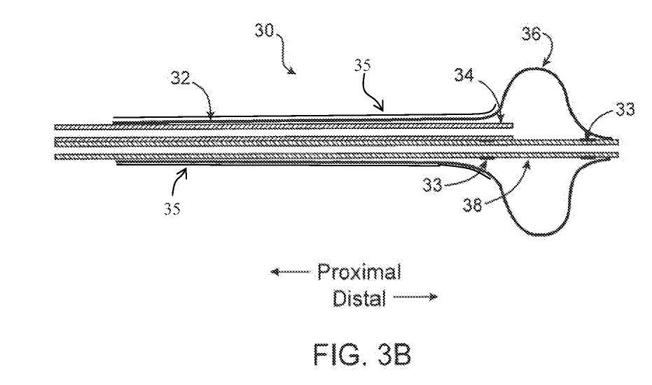

Referring now to FIGS. 3A and 3B, a distal portion of a catheter device 30 according to one embodiment is shown in perspective view and cross-sectional view, respectively. Catheter device 30 suitably includes a catheter body 32, which includes an expandable anchoring member 36, houses an inflation tube 34 and an injection tube 38, and has several radiopaque markers 33 disposed along its distal portion. Anchoring member 36 enables a distal portion of catheter device 30 to be maintained in a position within a disc. Inflation tube 34 is used to expand anchoring member 36, which in the embodiment shown comprises an expandable balloon. Injection tube 38 is used to introduce one or more fluids into the nucleus pulposus of the disc. Radiopaque markers 33 facilitate visualization of the distal portion of catheter device 30 so that its location may be assessed before, during or after a diagnostic or therapeutic procedure. In an exemplary embodiment, catheter body 32 is made from an optically conductive material that connects to the anchoring member 36 which is also made from an optically conductive material. In this embodiment, catheter body 32 has a surface coating 35 that inhibits discharge of the low level light energy, whereas the anchoring member 36 does not have such a coating and is configured to readily emit the low level light energy to the surrounding tissue. In another embodiment, the therapeutic energy is delivered to the anchoring member 36, which acts as a diffuser (see U.S. Pat. No. 6,846,098), by way of a wire or cable (not shown) running within the inflation tube 34 or injection tube 38.

In various embodiments, the distal portion of catheter device 30 may have one or more features that facilitate advancement of the distal portion through an annulus fibrosis of an intervertebral disc. A distal portion having one or more such features is generally referred to as “self-introducing.” Therefore, by “self-introducing” it is meant simply that the distal portion has one or more features for facilitating its passage through annulus fibrosis tissue. Such features may include, for example, one or more sections on a catheter shaft that are stiffer than adjacent sections to help make the shaft pushable. Another feature may comprise a tapered or pointed distal tip for piercing through annulus fibrosis. In some embodiments, catheter device 30 may be coupled with a removable, pointed stylet. These or any other suitable features may be included in a distal portion of catheter device 30 for facilitating passage through an annulus fibrosis.

The various components of catheter device 30 may be constructed from any suitable materials and may have any suitable shapes, sizes, dimensions or the like in various embodiments. In one embodiment, for example, the cross-sectional diameter of catheter body 32 decreases along its length from its proximal end to its distal end. Such a tapered configuration may allow catheter device 30 to be easily introduced through an introducer device. The outer diameter of catheter body 32 will also generally be slightly smaller than an inner diameter of an introducer device. In one embodiment, for example, catheter body 32, including any surface coating 35 that may be present, has an outer diameter of about 2 mm or less along at least part of its length.

In various embodiments, catheter body 32 may comprise a rigid single polymer or a composite consisting of reinforced metallic or polymeric components. Metallic components may include, for example, stainless steel, nitinol or other super-elastic alloys. Polymers may include, but are not limited to Polyetheretherketone (PEEK), Poly ether Block Amide (PEBAX), Nylon, Polyester, Polyolefin, polyamide, Polyimide, Polycarbonate, Polypropylene, Fluorinated Ethylene Polymer (FEP), Perfluoroalkoxy (PFA), Polytetrafluoroethylene-Perfluoromethylvinylether (MFA), Polyurethane, Low density polyethylene (LDPE), silicon dioxide (SiO₂), germanium tetrachloride (GeCl₄), phosphorus oxychloride (POCl₃), or may be made with two or more polymers, such as a polymer wall having an outer shell, or cladding (see U.S. Pat. Nos. 4,850,672; and 5,290,892). Such materials may be reinforced with coils or braids in some embodiments. The materials may also be coated internally or externally with materials the resist friction such as Teflon (Poly-Tetra-Fluoro-Ethylene), hydrophilic materials, parylene or the like.

In various embodiments, the therapeutic energy conduit may be an optical fiber and may comprise a transparent core surrounded by a cladding material and may be a multi-mode fiber or a single-mode fiber. The optic fiber or optically transparent material may be made from glass, including glass comprising silica, fluorozirconate, fluoroaluminate, and/or chalcogenide, crystalline material, including sapphire, quartz or diamond. See FABRICATION OF HOLLOW OPTICAL WAVEGUIDES ON PLANAR SUBSTRATES, John P. Barber, 2006, Doctorate Dissertation, Brigham Young University.

In various embodiments, catheter 30 may include one or more radiopaque markers 33 and/or may be made from one or more radiopaque materials to facilitate visualization. Such radiopaque markers/materials may include, but are not limited to, gold, Platinum, Iridium, Tungsten, Tantulum, resins containing Barium Sulfate, Bismuth trioxide or Tungsten and/or the like.

Anchoring member 36 may also be made of any suitable materials now known or discovered in the future, according to various embodiments. For example, expandable anchoring member 36 may comprise any transparent or semi-transparent material and a light emitting diode (LED) in optical communication therewith. Various adhesives may be used to attach anchoring member 36 to catheter shaft 32 or for any other suitable purpose. Any suitable adhesive(s) may be used, such as but not limited to, light activated acrylics, light activated cyanoacrylates, light activated silicones, heat activated adhesives, ambient curing adhesives, cyanoacrylates, epoxy adhesives, and/or polyurethane adhesives. Various parts of catheter device 30 may also be attached using alternative means, such as friction fitting, snap fitting, screw fitting, application of energy such as thermal or radio frequency energy, and/or the like.

In FIG. 3B anchoring member 36 is made of an optically transmitting material having a light blocking cladding 35 running from the proximal end of the catheter 30 to near the distal end of the catheter 30, such that the non-clad distal end of the anchoring member 36 also forms a therapeutic delivery surface.

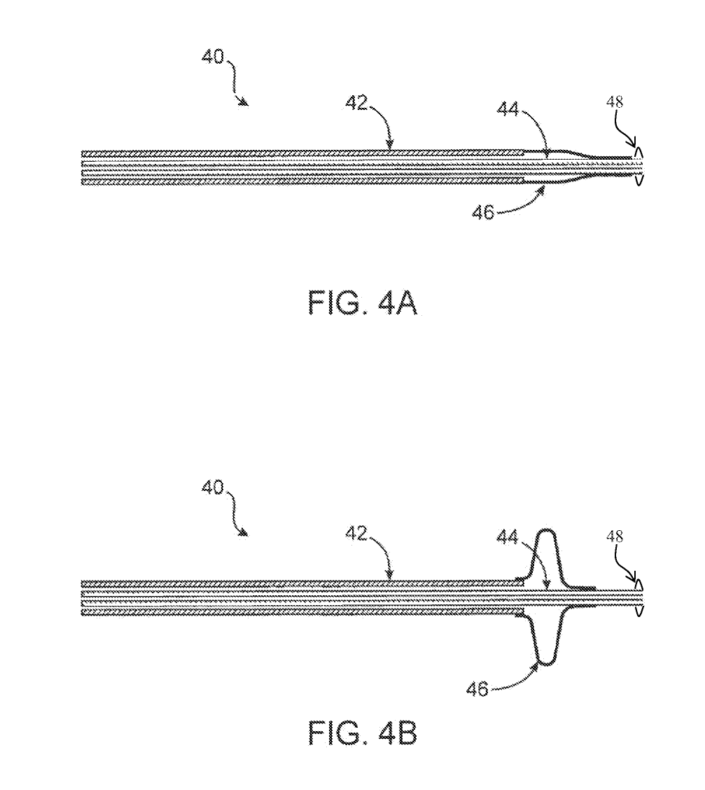

Referring now to FIGS. 4A and 4B, in another embodiment a catheter device 40 comprises an outer shaft 42, an inner shaft 44, and an anchoring member 46 coupled with both outer shaft 42 and inner shaft 44. Shafts 42, 44 are axially slidable relative to one another, such that when inner shaft 44 is moved proximally relative to outer shaft 42, anchoring member 46 buckles outward to perform its anchoring function, as shown in FIG. 4B. In this embodiment, inner shaft 44 acts as an injection lumen, and a therapeutic energy conduit and also possibly as a guidewire lumen, and no inflation lumen is needed. In one embodiment, a low level light diffuser 48 is located at the distal end of inner shaft 44 and the inner shaft 44 comprises an optic cable to transmit the low level light to the diffuser 48, which radiates the light out in about a 180 degree arch to the surrounding tissue. The low level light has an average power between about 1 mW to about 500 mW and irradiates the surrounding tissue at about 10 mW/cm² to about 5 W/cm². The typical wavelength is in the range 600-1000 nm. In one embodiment, anchoring member 46 may be constructed as a cylinder with slots or other shapes cut out of it to form colunm-like buckling structures. Components of this embodiment may be made of the same or different materials as just described.

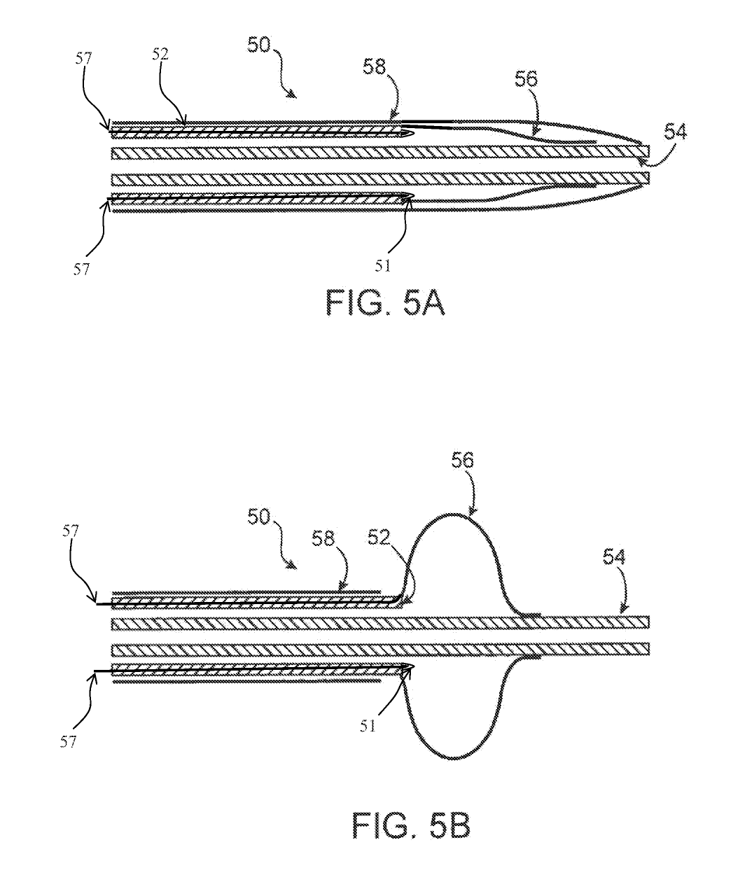

An alternative embodiment of a catheter device 50 is shown in FIGS. 5A and 5B. Here, catheter device 50 includes an outer shaft 52 having a therapeutic energy conduit 57 in electrical connection to one or more LEDs 51, an inner shaft 54, an anchoring member 56 that is configured to diffuse low level light generated by the LEDs 51 into the surrounding tissue and coupled to outer shaft 52 and inner shaft 54. A sheath 58 is slidably disposed over outer shaft 52. When sheath 58 is disposed over anchoring member 56, as in FIG. 5A, anchoring member 56 remains in an undeployed state suitable for delivery into the disc. When sheath 58 is retracted and/or outer shaft 52 is advanced, as in FIG. 5B, anchoring member 56 may be deployed and low level light from the energy conduit 57 and LED 51 may be emitted into the surrounding area. In some embodiments, anchoring member 56 may be deployed via buckling or via inflation, as described above.

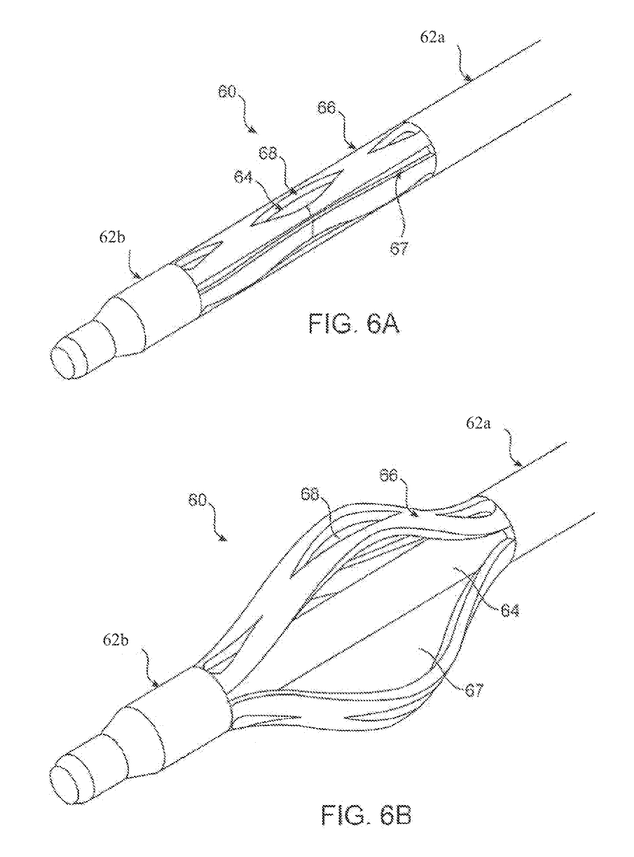

Referring now to FIGS. 6A and 6B, in another embodiment a catheter device 60 includes an outer shaft 62 having an expandable anchoring portion 66 and an inner shaft 64. Expandable anchoring portion 66 generally comprises a buckling portion of outer shaft 62 that acts as a low level light diffuser that may include multiple features, such as small cut-outs 68 and larger openings 67. When a proximal portion of outer shaft 62a is moved toward a distal portion of outer shaft 62b, anchoring member 66 buckles, due to features 67, 68, thus providing the anchoring function and configuring the therapeutic delivery surface so as to emit therapeutic energy from an arched surface, thereby delivering the energy more evenly to the surrounding tissue (see U.S. Pat. No. 6,398,778).

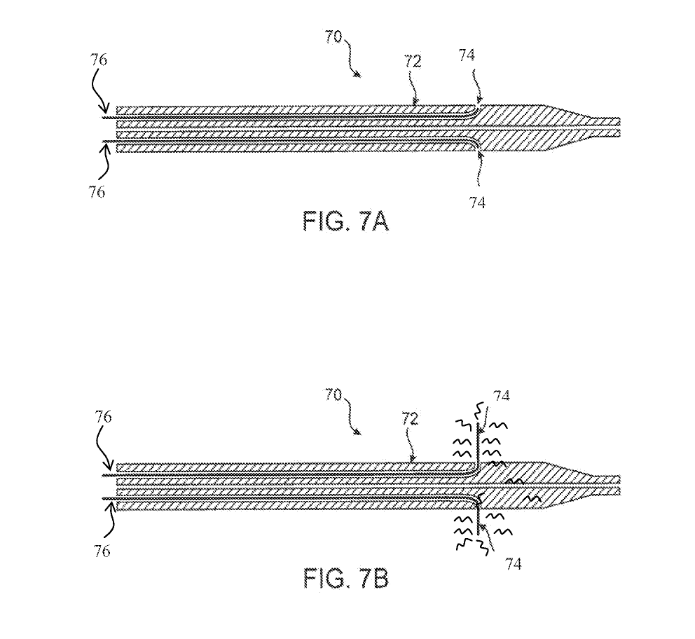

With reference now to FIGS. 7A and 7B, another embodiment of a catheter device 70 is illustrated having a catheter shaft 72 and retractable anchors 76. Anchors 76 may be deployed from a retracted state, as in FIG. 7A, to a deployed state, as in FIG. 7B. The anchors 76 comprise a therapeutic energy conduit running therethrough and an therapeutic delivery surface 74 located at the distal end. In various embodiments, the anchors 76 may be deployed either by pushing them out of their housing lumens or by releasing them from constraint to allow them to self-deploy.

In various embodiments, the anchoring member of a catheter device may have any suitable shape, size, configuration, orientation to the catheter shaft or the like.

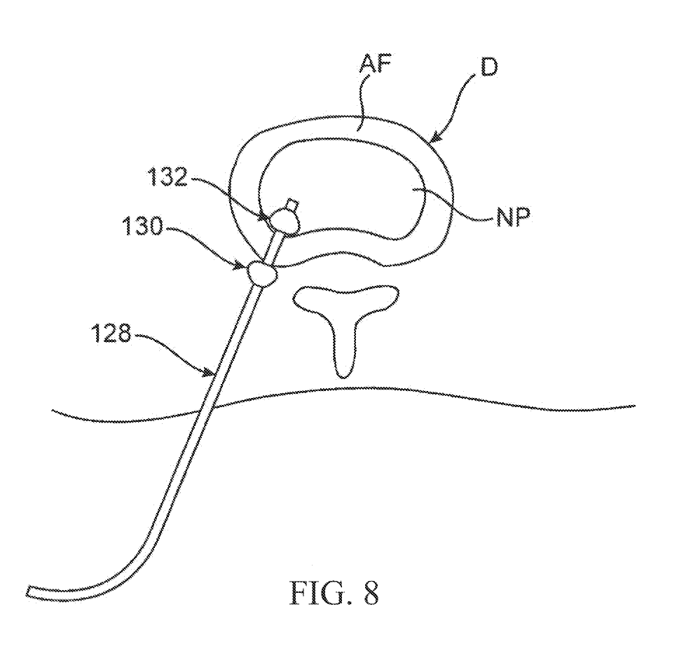

With reference now to FIG. 8, in another embodiment a catheter device 128 includes an outer anchoring member 130 for anchoring outside the annulus fibrosis AF and an inner anchoring member 132 for anchoring inside the disc D, typically in the nucleus pulposus NP. As shown here, anchoring members 130, 132 may comprise expandable members, such as inflatable balloons. In this embodiment, the inner and/or the outer anchoring member 130, 132 may comprise a therapeutic energy transmitting surface in communication with a therapeutic energy source, e.g., a LED or fiber optic cable. When both the inner anchoring member 130 and the outer anchoring member 132 comprise therapeutic energy delivery surfaces, the therapeutic energy may be effectively applied to the both the inner disc space, typically the nucleus pulposus NP, and the outer area, typically the outer surface of the annulus fibrosus AF and surrounding tissue.

In some embodiments the catheter is connected to a therapeutic energy source that may be programmed to deliver therapeutic energy, such as low level light, at a predetermined rate, at programmed intervals, upon triggering by the patient or physician through the use of an external device capable of communicating with the energy source, such as but not limited to magnetic reed switches, electromagnetic wave communication devices such as visible light, radio-wave, microwave, or short-wave, or wireless communication protocols such as Bluetooth.

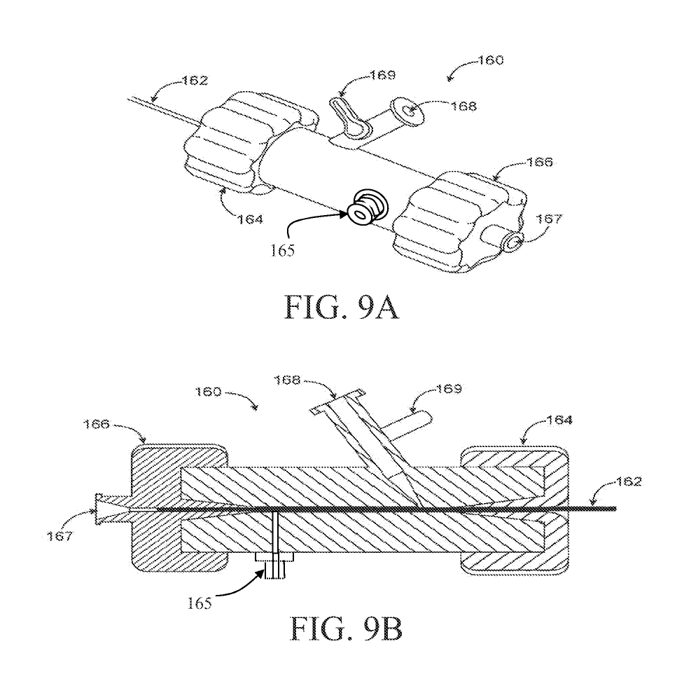

With reference to FIGS. 9A and 9B, one embodiment includes one or more adapters 160 for removably coupling with one or more proximal ends of a catheter device 162. Adapter 160 is typically coupled with catheter device 162 after the distal end of catheter device 162 is in place within the disc and after the introducer device, stylet or the like has been removed, although in alternative embodiments adapter 160 may be coupled with catheter device 162 at any other suitable time. Adapter 160 may comprise or resemble a Touhy Bourst adapter, compression fitting, instant tube fitting, or other similar adapter or connector. In one embodiment, adapter includes a distal sealed connector 164, a proximal sealed connector 166 having an injection port 167, an anchoring member inflation port 168 with a stopcock 169 for controlling fluid flow through inflation port 168 and a fiber optic connector 165. Fiber optic connector 165 is in optic communication with a therapeutic energy conduit in the catheter device 162 and may be used to connect the catheter device 162 to an energy source (not shown). The fiber optic connector 165 may include any method and connection systems that mate the ends of fiber optic cables in such a way that sufficient light is transmitted across the junction. Injection port 167 is in fluid communication with a lumen of catheter device 162 and may be used for injection of substance(s) into the disc and/or for passage of a guidewire. Injection port 167 and inflation port 168 may include luer fittings, press fits, barbs or any other suitable tube connection methods.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all publications, such as patents and patent applications, cited herein are hereby incorporated by reference in their entirety.

What is claimed is:

1. A method of treating pain, comprising the step of:
making an incision in a subject;
inserting a catheter having a light emitting surface located at a distal end into the incision;
advancing the distal end of the catheter to a position in or near the subject's spine thought to be responsible for generating pain in the subject;
irradiating the tissue at or near the distal end of the catheter with light having a wavelength of between 600 nm and 1000 nm in an amount of between 0.02 J/cm² and 200 J/cm²; and
removing the catheter from the subject.

2. The method of claim 1, advancing the distal end of the catheter to a position in an intervertebral disc and delivering light into the nucleus pulposus of the intervertebral disc.

3. The method of claim 2, further comprising irradiating the annulus fibrosus area either prior to inserting the catheter into the intervertebral disc or after removing the catheter from the intervertebral disc.

4. The method of claim 1, wherein the light is coupled with a catheter having one or more anchoring members.

5. The method of claim 1, wherein the light is administered in an amount of between about 1 J/cm² and about 10 J/cm².

6. The method of claim 1, wherein the area thought to be generating pain in the subject is a facet joint or a vertebra.

7. The method of claim 6, further comprising administering a corticosteroid and an analgesic.

8. The method of claim 1, further comprising administering at least one beneficial substance through the catheter to the area thought to be generating pain in the subject.

9. The method of claim 1, wherein the light is delivered to the surrounding tissue the entire time the catheter is being withdrawn from a subject.

10. The method of claim 1, comprising diffusely irradiating the light into the surrounding tissue through a therapeutic light emitting anchoring member.

11. A catheter instrument, comprising:
a hollow tube having a throughbore, a proximal end portion and a distal end portion;
a conduit disposed within the throughbore and running from the distal end of the tube to the proximal end;
an energy source connected to the conduit at the proximal end;
a light emitting diode (LED) disposed at the distal end of the conduit and in electrical communication therewith; and
an anchoring member made of a substantially light transparent material, wherein activation of the LED emits light through the anchoring member at a wavelength of between 600 nm and 1000 nm and in an amount of between 0.02 J/cm² and 200 J/cm².

12. The instrument of claim 11, wherein the throughbore comprises an injection lumen.

13. The instrument of claim 12, further comprising an inflation tube located in the throughbore and in inflatable communication with the anchoring member.

14. The instrument of claim 11, wherein the energy source comprises a battery power source.

15. A low level laser light emitting catheter, comprising:
a catheter having a throughbore with a proximal end and a distal end;
a light transparent material disposed near the distal end;
a fiber optic cable having a distal end connected to the light transparent material at the distal end of the catheter and a proximal end located near the proximal end of the catheter; and
a light source connected to the proximal end of the fiber optic cable.

16. The low level laser light emitting catheter of claim 15, wherein the light transparent material comprises an anchoring member.

17. The low level laser light emitting catheter of claim 16, wherein the light transparent material forms a light diffusing surface at the distal end of the catheter.

18. The low level laser light emitting catheter of claim 15, wherein the light source emits a light at a wavelength of between 600 nm and 1000 nm and in an amount of between 0.02 J/cm² and 200 J/cm².

19. The low level laser light emitting catheter of claim 15, wherein the throughbore comprises an injection lumen and an inflation tube connected to the anchoring member.
